(19) United States
(12) Patent Application Publication
(10) Pub. No.: US 2025/0205235 A1
(43) Pub. Date: Jun. 26, 2025

(54) DOSE REGIMEN FOR VERICIGUAT

(71) Applicant: ADVERIO PHARMA GMBH, Leverkusen (DE)

(72) Inventors: Stefano CORDA, Basel (CH); Corina BECKER, Düsseldorf (DE); Monica BRATTI, Berlin (DE); Monika SCHULZE ZUMLOH, Berlin (DE); Ciaran Joseph MCMULLAN, Rahway, NJ (US); Robert BLAUSTEIN, Rahway, NJ (US); Najwa MOSTAFA, Rahway, NJ (US)

(21) Appl. No.: 18/980,480

(22) Filed: Dec. 13, 2024

Related U.S. Application Data
(60) Provisional application No. 63/612,591, filed on Dec. 20, 2023.

(51) Int. Cl. A61K 31/506; A61K 9/00; A61K 45/06; A61P 9/04

(52) U.S. Cl. CPC A61K 31/506; A61K 9/0053; A61K 45/06; A61P 9/04

(57) ABSTRACT

A method of administering vericiguat or a pharmaceutically acceptable salt thereof during initiation of treatment comprising a two-dose regimen.

Methods for oral administration of methyl {4,6-diamino-2-[5-fluoro-1-(2-fluorobenzyl)-1H-pyrazolo[3,4-b]pyridin-3-yl]pyrimidin-5-yl}carbamate, also known as vericiguat, are disclosed. The methods concern dosage regimens that are safe and effective and their use to treat or prevent cardiovascular events in patients with heart failure and other disorders.

Verquvo® vericiguat was approved for sale in the U.S. as 2.5, 5, and 10 mg tablets to reduce the risk of cardiovascular death and heart failure (HF) hospitalization following a hospitalization for heart failure or need for outpatient IV diuretics, in adults with symptomatic chronic HF and ejection fraction less than 45%. Vericiguat has the chemical name methyl {4,6-diamino-2-[5-fluoro-1-(2-fluorobenzyl)-1H-pyrazolo[3,4-b]pyridin-3-yl]pyrimidin-5-yl}carbamate and has this chemical structure:

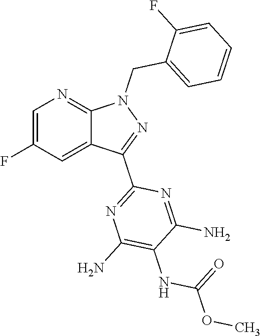 [SMILES: COC(=O)Nc1c(N)nc(-c2nn(Cc3ccccc3F)c3ncc(F)cc23)nc1N]

Vericiguat and processes for its preparation and use are disclosed in U.S. Pat. Nos. 8,420,656; 8,921,377; 9,604,948; 9,993,476; 10,736,896; and 11,439,642. It is known that dosing of Verquvo® vericiguat may result in hypotension. In clinical trials, some patients receiving vericiguat experienced a reduction of systolic blood pressure compared with placebo. As of June 2023, the US Food and Drug Administration (FDA) has recommended a starting dose of Verquvo® vericiguat of 2.5 mg orally once daily with food. Thereafter, the recommendation is to double the dosage of Verquvo® vericiguat approximately every two weeks to reach the target maintenance dosage of 10 mg orally once daily. Notwithstanding these starting dosage directions, many patients are not completing the dosage titration steps. Many patients are remaining on dosages that are below those proven efficacious longer than recommended, and, in some cases, patients are never moving to the therapeutically effective dose of 10 mg orally once daily but remain dosing at 2.5 mg or 5.0 mg daily due to errors in following the dosage recommendations and for other reasons. In a study of U.S. patients, among 2,765 patients treated with vericiguat (mean age 67.4 years, 68.1% male), only 34% reached the target dose of 10 mg. Victores et al., “Who?Characterizing vericiguat use in a real-world HFrEF patient population,” Abstract: 23-A-12830-ACC (Am. Coll. Cardiology Conf., Mar. 4-6, 2023, New Orleans, LA, 2023. Kerwagen et al. reported that in Germany during the 16-month study period since the launch of vericiguat in September 2021, initiation of vericiguat was observed in 2,916 (median age 75 years, 72% male) patients. Kerwagen et al., “Indicators of adherence and persistence to vericiguat in Germany: a prescription claims data analysis,” DGK Herztage, Bonn. Clin Res Cardiol. 2023; 112:1713, doi.org/10.1007/s00392-023-02302-4. During a median follow-up of 150 days, 33% of the patients reached the 5 mg, and 36% reached the 10 mg dose. In a retrospective cohort study using a Japanese hospital administrative database, patient characteristics at the initiation of vericiguat treatment, patterns of HF medication use, and vericiguat dose titrations were assessed within the first 90 days of treatment. The study included 829 patients who were initiated on vericiguat therapy. Most patients had previously received HF medications, with high percentages using angiotensin-receptor blocker neprilysin inhibitors (ARNI; 43.9%) and sodium glucose cotransporter-2 inhibitors (54.4%). During the first 90 days of vericiguat treatment, 65.8% of the patients were up titrated from their starting dose, and 32.3% had reached the maximal daily dose. The median time to reach the maximal daily dose was 34 days. Okami et al. “Vericiguat Use in Patients with Heart Failure in Real-World Settings during the First Year after the Drug Authorization in Japan”. J. Clin. Med. 2024, 13(11), 3222. This real-world data shows the clinical need to achieve the 10 mg target dose of vericiguat in a safe and faster manner.

Thus, a need exists for an advantageous vericiguat dosing regimen for patients who are initiating treatment that reduces the number of patients who spend an unnecessary period on suboptimal vericiguat dosages including reduces the number of patients who fail to titrate up to the full efficacious dose. There exists a need for a dosage regimen for initiating vericiguat patients that improves patient compliance in titrating up to a therapeutically effective dosage, without an unacceptable risk of symptomatic hypotension or another adverse event.

Vericiguat acts as a stimulator of soluble guanylate cyclase (sGC), which is a key enzyme of the nitric oxide (NO) signaling pathway. On binding of NO to a prosthetic heme group on sGC, sGC catalyzes synthesis of the second messenger cGMP, which produces vasorelaxation and inhibits smooth muscle proliferation, leukocyte recruitment, and platelet aggregation through a number of downstream mechanisms. Because of these effects, sGC is a therapeutic target in cardiovascular disorders. See Stasch et al., “Soluble Guanylate Cyclase as an Emerging Therapeutic Target in Cardiopulmonary Disease,” Circulation, 123 (20) (24 May 2011).

Starting dosage regimens have been reported for other sGC stimulators. Adempas® riociguat was the first sGC stimulator to be approved in the U.S. It is approved to treat chronic thromboembolic pulmonary hypertension (CTEPH) and pulmonary arterial hypertension. As of June 2023, the Adempas® riociguat prescribing information instructs that a new patient start at a dosage of 1 mg taken three times a day. For patients who cannot tolerate the hypotensive effect of Adempas® riociguat, the recommendation is a starting dose of 0.5 mg taken three times a day. If systolic blood pressure remains greater than 95 mmHg and the patient has no signs or symptoms of hypotension, then the recommendation is to up-titrate the dose by 0.5 mg taken three times a day. Dose increases are taught to be no sooner than two weeks apart. The dose can be increased to the highest tolerated dosage, up to a maximum of 2.5 mg taken three times a day. The Adempas® riociguat 2023 prescribing information further states that if at any time the patient has symptoms of hypotension, then dosage should be decreased by 0.5 mg taken three times a day. In case Adempas® riociguat is interrupted for three days or more, then the patient should be re-titrated with Adempas® riociguat. Because Adempas® riociguat reduces blood pressure, the labelling recommends that physicians consider the potential for symptomatic hypotension or ischemia.

S. Katta et al. reported rapid titration of riociguat in approximately four days in thirteen patients. Am J Respir Crit Care Med 2020; 201:A2042 and Am J Respir Crit Care Med 2019; 199:A1999.

Another sGC stimulator is praliciguat, chemical name 1,1,1,3,3,3-hexafluoro-2-[[[5-fluoro-2-[1-[(2-fluorophenyl)methyl]-5-(1,2-oxazol-3-yl)pyrazol-3-yl]pyrimidin-4-yl]amino]methyl]propan-2-ol, which is now in clinical trials. Hanrahan et al., Diabetes 2018; 67(Supplement_1):1207-P, reports a rapid dose escalation study of praliciguat in eleven patients with type 2 diabetes and hypertension. Buys et al., Nitric Oxide 78 (2018) 72-80, Elsevier Inc.

In the context of the present disclosure, “dose regimen” and “dosage regimen” are used synonymously and include the meaning of “titration regimen”.

In the context of the present disclosure, “initiation of treatment” is defined as administering vericiguat to a patient who has never before received vericiguat.

In the context of the present disclosure, “starting dose”, “initial dose”, and “initiation dose” are defined as the dose of vericiguat administered to a patient who has never before received vericiguat.

In the context of the present disclosure, “target maintenance dose” is defined as a dose of Verquvo® vericiguat of 10 mg once daily, as tolerated by the patient.

In the context of the present disclosure, “temporary interruption of treatment or study intervention” is defined as an interruption of treatment or study intervention of 1 or 2 days during the first treatment cycle.

In the context of the present disclosure, “symptomatic hypotension of moderate intensity” is defined as a type of adverse event that is usually alleviated with additional specific therapeutic intervention. The event interferes with usual activities of daily living, causing discomfort but poses no significant or permanent risk of harm to the research participant.

In the context of the present disclosure, “symptomatic hypotension of severe intensity” is defined as a type of adverse event that interrupts usual activities of daily living, or significantly affects clinical status, or may require intensive therapeutic intervention.

In the context of the present disclosure, “recent history of symptomatic hypotension” is defined as history of symptomatic hypotension 4 weeks before screening.

In the context of the present disclosure, “ejection fraction” is used synonymously with Left Ventricular Ejection Fraction (LVEF).

According to some aspects the disclosure concerns a method of administering vericiguat or a pharmaceutically acceptable salt thereof, to a human subject in need thereof for use in treatment of cardiovascular and pulmonary disorders, comprising the following steps: during initiation of treatment, administering vericiguat, or a pharmaceutically acceptable salt thereof, orally once daily as follows: about 5 mg on days 1 to 14; about 10 mg on days 15 to 28. In another aspect, the disclosure concerns a method of administering vericiguat or a pharmaceutically acceptable salt thereof, to a human subject in need thereof for use in treatment of cardiovascular and pulmonary disorders, comprising the following steps: during initiation of treatment, administering vericiguat, or a pharmaceutically acceptable salt thereof, in a dosing regimen comprising a first treatment cycle followed immediately by a second treatment cycle, wherein said first treatment cycle comprises orally administering about 5 mg for between 9-19 consecutive days and said second treatment cycle comprises orally administering about 10 mg for at least 14 consecutive days.

According to another aspect the disclosure concerns a method of reducing the risk of cardiovascular death and heart failure hospitalization in a human subject with symptomatic chronic heart failure by administering vericiguat comprising the following steps: during initiation of vericiguat treatment, administering vericiguat in a dosing regimen comprising a first treatment cycle followed immediately by a second treatment cycle, wherein said first treatment cycle comprises orally administering 5 mg once daily with food for approximately two weeks and said second treatment cycle comprises orally administering 10 mg once daily with food. In some embodiments, the second treatment cycle comprises orally administering 10 mg once daily with food for at least about two weeks.

Another aspect of the disclosure is a kit of parts comprising:
(a) a compound that is vericiguat or a pharmaceutically acceptable salt thereof; and
(b) instructions for administering the compound in a dosing regimen, wherein the dosing regimen is selected from the group consisting of
(i) during initiation of treatment, administering the compound orally once daily as follows: about 5 mg on days 1 to 14; about 10 mg on days 15 to 28;
(ii) during initiation of treatment, administering the compound in a dosing regimen comprising a first treatment cycle followed immediately by a second treatment cycle, wherein said first treatment cycle comprises orally administering about 5 mg for between 9-19, preferably 14, consecutive days and said second treatment cycle comprises orally administering about 10 mg for at least 14 consecutive days; and
(iii) during initiation of treatment, administering vericiguat in a dosing regimen comprising a first treatment cycle followed immediately by a second treatment cycle, wherein said first treatment cycle comprises orally administering 5 mg once daily with food for approximately two weeks and said second treatment cycle comprises orally administering 10 mg once daily with food. In some embodiments, the second treatment cycle comprises orally administering 10 mg once daily with food for at least about two weeks.

In another aspect, the disclosure concerns a method of reducing the risk of cardiovascular death and heart failure hospitalization in a population of human patients with chronic heart failure and ejection fraction less than 45%, comprising administering to each human patient in the population vericiguat or a pharmaceutically acceptable salt thereof following a dosing regimen during initiation of treatment, wherein the dosing regimen comprises a first treatment cycle followed immediately by a second treatment cycle, wherein said first treatment cycle comprises orally administering about 5 mg, preferably 5 mg, for between 9-19, preferably 14, consecutive days and said second treatment cycle comprises orally administering about 10 mg, preferably 10 mg, for at least 14 consecutive days; and wherein the tolerability of the patient population is at least 90%, or at least 93%.

In the context of the present invention, physiologically acceptable salts of vericiguat include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, formic acid, acetic acid, trifluoroacetic acid, propionic acid, lactic acid, tartaric acid, malic acid, citric acid, fumaric acid, maleic acid and benzoic acid. Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Vericiguat and methods of its manufacture and use are disclosed in U.S. Pat. No. 8,420,656. The present disclosure includes preventing and/or treating disorders including the following: the treatment and/or prophylaxis of cardiovascular, pulmonary, and thromboembolic disorders. Some examples of cardiovascular disorders that may be treated or prevented with vericiguat are, without limitation, hypertension, acute and chronic heart failure, coronary heart disease, stable and unstable angina pectoris, and peripheral and cardiac vascular disorders.

In the context of the present disclosure, the term heart failure also includes more specific or related types of disease, such as acute decompensated heart failure, right heart failure, left heart failure, global failure, ischaemic cardiomyopathy, dilated cardiomyopathy, hypertrophic cardiomyopathy, idiopathic cardiomyopathy, congenital heart defects, heart valve defects, heart failure associated with heart valve defects, mitral stenosis, mitral insufficiency, aortic stenosis, aortic insufficiency, tricuspid stenosis, tricuspid insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, heart failure with reduced ejection fraction (HFrEF) and heart failure with preserved ejection fraction (HFpEF). Chronic heart failure patients having left ventricular ejection fraction (LVEF) of less than or equal to 40% are included in the patients treated by the present methods.

The disclosure also concerns the treatment and/or prophylaxis of kidney disorders, in particular of acute and chronic renal insufficiency and acute and chronic renal failure.

Furthermore, the disclosure also concerns the treatment and/or prophylaxis of pulmonary arterial hypertension (PAH) and other forms of pulmonary hypertension (PH) including left-heart disease and thromboembolisms including, without limitation, chronic thromboembolic pulmonary hypertension (CTEPH).

The disclosure also involves controlling central nervous system diseases characterized by disturbances of the NO/cGMP system. The methods are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, demyelination, multiple sclerosis, thalamic degeneration, Creutzfeld-Jacob dementia, HIV dementia, schizophrenia with dementia or Korsakoffs psychosis.

As used herein “patient” refers to a human subject, in some embodiments an adult human subject.

Vericiguat or a pharmaceutically acceptable salt thereof can be employed alone or in combination with other active agents. In one embodiment, the other active agents are one or more active agents that are used in the standard of care in contemporary guidelines for the disease afflicting the patient. For example, the patient may receive vericiguat or a pharmaceutically acceptable salt thereof in combination with the Guideline-directed medical therapy (GDMT), which is a form of treatment for heart failure with reduced ejection fraction. Doctors prescribe a combination of drugs to treat people's different symptoms. The GDMT may include one or more of the following active agents: beta-blockers; angiotensin-converting enzyme inhibitors (ACEI)/angiotensin receptor blockers (ARB)/angiotensin receptor-neprilysin inhibitors (ARNI); mineralocorticoid receptor antagonists (MRA); and sodium-glucose cotransporter-2 inhibitors (SGLT2i). One or more of these active agents from the GDMT may be co-administered with vericiguat or a pharmaceutically acceptable salt thereof under the instantly disclosed dosage regimens. In one embodiment, the human subject is administered vericiguat according to any of the dosage regimens herein and concomitantly is administered one, two, three, four, or five further active agents selected from the list consisting of beta-blockers; angiotensin-converting enzyme inhibitors (ACEI)/angiotensin receptor blockers (ARB)/angiotensin receptor-neprilysin inhibitors (ARNI); mineralocorticoid receptor antagonists (MRA); and sodium-glucose cotransporter-2 inhibitors (SGLT2i). The SGLT2i may be selected from the group consisting of dapagliflozin, canagliflozin, empagliflozin, ertugliflozin, remogliflozin, and sergliflozin. The beta blocker may be selected from the group consisting of atenolol, bisoprolol, carvedilol, metoprolol, nebivolol, and propranolol.

The present disclosure concerns oral administration of vericiguat or a pharmaceutically acceptable salt thereof using any suitable formulation for oral administration. Suitable administration forms for oral administration include those that work according to the prior art that provide immediate release of the compound, for example tablets (uncoated or coated tablets), tablets or films/wafers which disintegrate rapidly in the oral cavity, films/lyophilizates or capsules (for example hard or soft gelatin capsules), sugar-coated tablets, granules, pellets, powders, emulsions, suspensions, aerosols or solutions. In one preferred aspect, vericiguat or a pharmaceutically acceptable salt thereof is formulated in tablets that are film-coated.

For example a suitable tablet of vericiguat for oral administration may be the following:

### Composition:

100 mg vericiguat, 50 mg of lactose (monohydrate), 50 mg of maize starch (native), 10 mg of polyvinylpyrrolidone (PVP 25) (BASF, Ludwigshafen, Germany) and 2 mg of magnesium stearate.

Tablet weight 212 mg, diameter 8 mm, radius of curvature 12 mm.

### Preparation:

The mixture of vericiguat, lactose and starch is granulated with a 5% solution (w/w) of the PVP in water. After drying, the granules are mixed with the magnesium stearate for 5 minutes. This mixture is pressed with a conventional tableting press (for tablet dimensions see above). The guide value used for the pressing is a pressing force of 15 kN.

### Dosage Regimen

The present disclosure concerns a dosage regimen to be used administering vericiguat to a patient who has never before received vericiguat (“initiating” treatment with vericiguat).

In one aspect, the patient is administered vericiguat, or a pharmaceutically acceptable salt thereof, orally once daily as follows: about 4, 5, or 6 mg on days 1 to 14; about 9, 10, or 11 mg on days 15 to 28. In another aspect the patient is administered vericiguat, or a pharmaceutically acceptable salt thereof, orally once daily as follows: 5.0 mg on days 1 to 14; 10.0 mg on days 15 to 28.

In one aspect, the dosage regimen comprises a first treatment cycle followed immediately by a second treatment cycle, wherein said first treatment cycle comprises orally administering about 5 mg, at least 5 mg, 5 mg, 3-5 mg, 4-5 mg, or 5-6 mg for between 9-19, 9-10, 9-11, 9-12, 9-13, 9-14, 9-15, 9-16, 9-17, 9-18, 10-11, 10-12, 10-13, 10-14, 10-15, 10-16, 10-17, 10-18, 10-19, 11-12, 11-13, 11-14, 11-15, 11-16, 11-17, 11-18, 11-19, 12-13, 12-14, 12-15, 12-16, 12-17, 12-18, 12-19, 13-14, 13-15, 13-16, 13-17, 13-18, 13-19, 14-15, 14-16, 14-17, 14-18, 14-19, 15-16, 15-17, 15-18, 15-19, 16-17, 16-18, 16-19, 17-18, 17-19, 18-19, 10-18, 11-17, 12-16, or 13-15 consecutive days or for 9, 10, 11, 12, 13, 14, 15, 16, 17, 18, or 19 consecutive days, or for approximately two weeks. The second treatment cycle comprises orally administering about 10 mg, at least 10 mg, 10 mg, 8-10 mg, 9-10 mg, or 10-11 mg. In some aspects, the second treatment cycle is at least 10, 11, 12, 13, 14, 15, 16, 17, 18, 19, 20, or 21 consecutive days, or approximately two weeks. “Followed immediately” as used herein means with no more than about 1 day, and preferably no days, between the first and the second treatment cycle. “About” as used herein for a dosage amount means plus or minus 5-10% of the recited numerical value. Each of the dosages of vericiguat or a pharmaceutically acceptable salt thereof used herein is understood to be a total daily dosage of the compound.

In one aspect, the dosage regimen does not result in an interruption of treatment. In another aspect, the dosage regimen does not result in an interruption of treatment during the first treatment cycle. An interruption of treatment as used herein refers to an interruption due to medical reasons associated with the administration of vericiguat or a pharmaceutically acceptable salt thereof, which may be symptomatic hypotension or another adverse event, particularly symptomatic hypotension. An interruption of treatment may be for 1-3 days, or 1, 2, or 3 days, or more than 1, more than 2 or more than 3 days. According to one embodiment, an interruption of treatment may be for 1 or 2 days.

### Kits

In another aspect, the present disclosure concerns a kit of parts comprising: (i) a compound that is vericiguat or a pharmaceutically acceptable salt thereof; and (ii) instructions for administering the compound in a dosing regimen. Any of the dosing regimens discussed herein may be the dosing regimen accompanying the compound in the kit. Preferably, the compound is in tablet form in the kit. The kit may also comprise a further active agent, such as one or more from the group of additional therapeutically active agents discussed herein.

In another aspect, the method of administration disclosed herein results in at least 90% or at least 93% of the patient population successfully completing the dosage regimen without treatment interruption due to medical effects of the vericiguat or pharmaceutically acceptable salt thereof. In one embodiment, treatment interruption is due to systemic hypotension. In one embodiment, treatment interruption is due to moderate to severe symptomatic hypotension associated with vericiguat. In another embodiment, treatment interruption is due to a medical provider recommending that vericiguat be stopped due to adverse events, including, without limitation, symptomatic hypotension or another adverse effect of vericiguat or the pharmaceutically acceptable salt thereof. In one embodiment, 100 or more patients are started on vericiguat by being administered vericiguat using one of the dosage regimens described herein and 90% or more or 93% or more of the patients tolerate the dosage regimen such that no stoppage of dosaging is medically indicated. In measuring whether the dosage is tolerated, adverse events such as moderate to severe symptomatic hypotension are determined. In one embodiment, the patient population consists of patients having HFrEF, EF<45% (assessed within 12 months before Visit 1 by any imaging method), preferably who are hemodynamically stable. In other embodiments, 80%, 81%, 82%, 83%, 84%, 85%, 86%, 87%, 88%, 89%, 90%, 91%, 92%, 93%, 94%, 95%, 96%, 97%, 98%, 99% or more, or 100%, of the patients tolerate the dosage regimen such that no stoppage of dosaging is medically indicated, such as when no symptomatic hypotension of moderate to severe nature is observed. All patient cases exceeding one missing dose are not counted for the analysis of the primary endpoint, which is the tolerability of 5 mg as a starting dosage of vericiguat.

In an embodiment, the patient population has symptomatic chronic heart failure with reduced ejection fraction (HFrEF), in another embodiment the patient population has symptomatic chronic heart failure with reduced ejection fraction (HFrEF) and the ejection fraction (EF)<45% (assessed within 12 months before Visit 1 by any imaging method).

### List of Abbreviations

| | |
|---|---|
| ACEI | Angiotensin-converting enzyme inhibitors |
| AE | Adverse Event |
| ARB | Angiotensin receptor blockers |
| ARNI | Angiotensin receptor-neprilysin inhibitors |
| CTEPH | Chronic thromboembolic pulmonary hypertension |
| EF | Ejection fraction |
| eGFR | Estimated glomerular filtration rate |
| FDA | Food and Drug Administration |
| GDMT | Guideline-directed medical therapy |
| HF | Heart failure |
| HFpEF | Heart failure with preserved ejection fraction |
| HFrEF | Heart failure with reduced ejection fraction |
| IV | Intravenous |
| LVEF | Left ventricular ejection fraction |
| MRA | Mineralocorticoid receptor antagonists |
| NO | Nitric oxide |
| PAH | Pulmonary arterial hypertension |
| SAE | Severe Adverse Event |
| SAF | Safety Analysis Set |
| SBP | Systolic blood pressure |
| sGC | Soluble guanylate cyclase |
| SGLT2i | sodium-glucose cotransporter-2 inhibitors |

### Embodiment 1

1. A method of administering vericiguat or a pharmaceutically acceptable salt thereof, to a human subject in need thereof for use in treatment of cardiovascular and pulmonary disorders, comprising the following steps: during initiation of treatment, administering vericiguat, or a pharmaceutically acceptable salt thereof, in a dosing regimen comprising a first treatment cycle followed immediately by a second treatment cycle, wherein said first treatment cycle comprises orally administering about 5 mg for between 9-19 consecutive days and said second treatment cycle comprises orally administering about 10 mg for at least 14 consecutive days.
2. The method of embodiment 1, wherein said first treatment cycle comprises orally administering at least 5 mg for between 9-19 consecutive days and said second treatment cycle comprises orally administering at least 10 mg for at least 14 consecutive days.
3. The method of embodiment 2, wherein said first treatment cycle comprises orally administering 5 mg for between 9-19 consecutive days and said second treatment cycle comprises orally administering 10 mg for at least 14 consecutive days.
4. The method of embodiment 1, wherein said first treatment cycle comprises orally administering at least 5 mg for 14 consecutive days and said second treatment cycle comprises orally administering at least 10 mg for at least 14 consecutive days.
5. The method of embodiment 3, wherein said first treatment cycle comprises orally administering 5 mg for 14 consecutive days and said second treatment cycle comprises orally administering 10 mg for at least 14 consecutive days.
6. A method of administering vericiguat or a pharmaceutically acceptable salt thereof, to a human subject in need thereof for use in treatment of cardiovascular and pulmonary disorders, comprising the following steps: during initiation of treatment, administering vericiguat, or a pharmaceutically acceptable salt thereof, orally once daily as follows: about 5 mg on days 1 to 14; about 10 mg on days 15 to 28.
7. The method of embodiment 6; wherein vericiguat, or a pharmaceutically acceptable salt thereof, is administered orally once daily as follows: 5.0 mg on days 1 to 14; 10.0 mg on days 15 to 28.
8. A method of reducing the risk of cardiovascular death and heart failure hospitalization in a human subject with symptomatic chronic heart failure by administering vericiguat comprising the following steps: during initiation of vericiguat treatment, administering vericiguat in a dosing regimen comprising a first treatment cycle followed immediately by a second treatment cycle, wherein said first treatment cycle comprises orally administering 5 mg once daily with food for approximately two weeks and said second treatment cycle comprises orally administering 10 mg once daily with food.
9. The method of embodiment 8, wherein said second treatment cycle comprises orally administering 10 mg once daily with food for at least two weeks.
10. The method of embodiment 1, 2, 3, 4, 5, 6, 7, 8, or 9, wherein the human subject has symptomatic chronic heart failure and ejection fraction less than 45% upon initiation of vericiguat administration.
11. The method of embodiment 1, 2, 3, 4, 5, 6, or 7, wherein the treatment of cardiovascular and pulmonary disorders results in reduced risk of cardiovascular death or heart failure hospitalization.
12. The method of embodiment 1, 2, 3, 4, 5, 6, or 7, wherein the treatment of cardiovascular and pulmonary disorders results in a reduced risk of a composite endpoint of cardiovascular death or heart failure hospitalization.
13. The method of embodiment 1, 2, 3, 4, 5, 6, 7, or 8, wherein vericiguat is administered and upon initiation of vericiguat administration the human subject has symptomatic chronic heart failure and ejection fraction less than 45% and has had no hospitalization within six months for heart failure and no outpatient IV diuretic use within three months.
14. The method of embodiment 1, 2, 3, 4, 5, 6, 7, 8, or 9, further comprising administering a further therapeutic agent selected from the group consisting of beta-blockers, angiotensin-converting enzyme inhibitors (ACEI), angiotensin receptor blockers (ARB), angiotensin receptor-neprilysin inhibitors (ARNI), mineralocorticoid receptor antagonists (MRA), and sodium-glucose cotransporter-2 inhibitors (SGLT2i).
15. The method of embodiment 1, 2, 3, 4, 5, 6, or 7, wherein vericiguat is administered.
16. A kit of parts comprising:
   (a) a compound that is vericiguat or a pharmaceutically acceptable salt thereof; and
   (b) instructions for administering the compound in a dosing regimen;
   wherein the dosing regimen is selected from the group consisting of
   (i) during initiation of treatment, administering the compound orally once daily as follows: about 5 mg on days 1 to 14; about 10 mg on days 15 to 28;
   (ii) during initiation of treatment, administering the compound in a dosing regimen comprising a first treatment cycle followed immediately by a second treatment cycle, wherein said first treatment cycle comprises orally administering about 5 mg for between 9-19, preferably 14, consecutive days and said second treatment cycle comprises orally administering about 10 mg for at least 14 consecutive days; and
   (iii) during initiation of treatment, administering vericiguat in a dosing regimen comprising a first treatment cycle followed immediately by a second treatment cycle, wherein said first treatment cycle comprises orally administering 5 mg once daily with food for approximately two weeks and said second treatment cycle comprises orally administering 10 mg once daily with food.
17. A method of reducing the risk of cardiovascular death and heart failure hospitalization in a population of human patients with chronic heart failure, comprising administering to each human patient in the population vericiguat or a pharmaceutically acceptable salt thereof following a dosing regimen during initiation of treatment,
   wherein the dosing regimen comprises a first treatment cycle followed immediately by a second treatment cycle,
   wherein said first treatment cycle comprises orally administering about 5 mg, preferably 5 mg, for between 9-19, preferably 14, consecutive days and said second treatment cycle comprises orally administering about 10 mg, preferably 10 mg, for at least 14 consecutive days; and
   wherein the tolerability of the patient population is at least 90%.
18. The method of embodiment 17, wherein tolerability is measured by the percentage of patients who do not experience an adverse drug reaction that results in interruption of treatment within the first treatment cycle.
19. The method of embodiment 18, wherein the adverse drug reaction is symptomatic hypotension.
20. The method of embodiment 17, 18 or 19, wherein the human patients in the population have chronic heart failure with an ejection fraction of less than 45%.

### Further Embodiment

1. A method of administering vericiguat or a pharmaceutically acceptable salt thereof, to a human subject in need thereof for use in treatment of cardiovascular and pulmonary disorders, comprising the following steps: during initiation of treatment, administering vericiguat, or a pharmaceutically acceptable salt thereof, in a dosing regimen comprising a first treatment cycle followed immediately by a second treatment cycle, wherein said first treatment cycle comprises orally administering about 5 mg for between 9-19 consecutive days and said second treatment cycle comprises orally administering about 10 mg for at least 14 consecutive days.
2. The method of embodiment 1, wherein said first treatment cycle comprises orally administering at least 5 mg for between 9-19 consecutive days and said second treatment cycle comprises orally administering at least 10 mg for at least 14 consecutive days.
3. The method of embodiment 2, wherein said first treatment cycle comprises orally administering 5 mg for between 9-19 consecutive days and said second treatment cycle comprises orally administering 10 mg for at least 14 consecutive days.
4. The method of embodiment 1, wherein said first treatment cycle comprises orally administering at least 5 mg for 14 consecutive days and said second treatment cycle comprises orally administering at least 10 mg for at least 14 consecutive days.
5. The method of embodiment 3, wherein said first treatment cycle comprises orally administering 5 mg for 14 consecutive days and said second treatment cycle comprises orally administering 10 mg for at least 14 consecutive days.
6. A method of administering vericiguat or a pharmaceutically acceptable salt thereof, to a human subject in need thereof for use in treatment of cardiovascular and pulmonary disorders, comprising the following steps: during initiation of treatment, administering vericiguat, or a pharmaceutically acceptable salt thereof, orally once daily as follows: about 5 mg on days 1 to 14; about 10 mg on days 15 to 28.
7. The method of embodiment 6; wherein vericiguat, or a pharmaceutically acceptable salt thereof, is administered orally once daily as follows: 5.0 mg on days 1 to 14; 10.0 mg on days 15 to 28.
8. A method of reducing the risk of cardiovascular death and heart failure hospitalization in a human subject with symptomatic chronic heart failure by administering vericiguat comprising the following steps: during initiation of vericiguat treatment, administering vericiguat in a dosing regimen comprising a first treatment cycle followed immediately by a second treatment cycle, wherein said first treatment cycle comprises orally administering 5 mg once daily with food for approximately two weeks and said second treatment cycle comprises orally administering 10 mg once daily with food.
9. The method of embodiment 8, wherein said second treatment cycle comprises orally administering 10 mg once daily with food for at least two weeks.
10. The method of embodiment 1, 2, 3, 4, 5, 6, 7, 8, or 9, wherein the human subject has symptomatic chronic heart failure and ejection fraction less than 45% upon initiation of vericiguat administration.
11. The method of embodiment 1, 2, 3, 4, 5, 6, or 7, wherein the treatment of cardiovascular and pulmonary disorders results in reduced risk of cardiovascular death or heart failure hospitalization.
12. The method of embodiment 1, 2, 3, 4, 5, 6, or 7, wherein the treatment of cardiovascular and pulmonary disorders results in a reduced risk of a composite endpoint of cardiovascular death or heart failure hospitalization.
13. The method of embodiment 1, 2, 3, 4, 5, 6, 7, or 8, wherein vericiguat is administered and upon initiation of vericiguat administration the human subject has symptomatic chronic heart failure and ejection fraction less than 45% and has had no hospitalization within six months for heart failure and no outpatient IV or SC diuretic use within three months.
14. The method of embodiment 1, 2, 3, 4, 5, 6, 7, or 8, wherein vericiguat is administered and upon initiation of vericiguat administration the human subject has symptomatic chronic heart failure and ejection fraction less than 45% and has had hospitalization within six months for heart failure or outpatient IV or SC diuretic use within three months.
15. The method of embodiment 1, 2, 3, 4, 5, 6, 7, 8, or 9, further comprising administering a further therapeutic agent selected from the group consisting of beta-blockers, angiotensin-converting enzyme inhibitors (ACEI), angiotensin receptor blockers (ARB), angiotensin receptor-neprilysin inhibitors (ARNI), mineralocorticoid receptor antagonists (MRA), and sodium-glucose cotransporter-2 inhibitors (SGLT2i).
16. The method of embodiment 1, 2, 3, 4, 5, 6, 7, 8, or 9, further comprising administering the further therapeutic agents angiotensin receptor-neprilysin inhibitors (ARNI) and sodium-glucose cotransporter-2 inhibitors (SGLT2i).
17. The method of embodiment 1, 2, 3, 4, 5, 6, or 7, wherein vericiguat is administered.
18. A kit of parts comprising:
   (a) a compound that is vericiguat or a pharmaceutically acceptable salt thereof; and
   (b) instructions for administering the compound in a dosing regimen;
   wherein the dosing regimen is selected from the group consisting of
   (i) during initiation of treatment, administering the compound orally once daily as follows: about 5 mg on days 1 to 14; about 10 mg on days 15 to 28;
   (ii) during initiation of treatment, administering the compound in a dosing regimen comprising a first treatment cycle followed immediately by a second treatment cycle, wherein said first treatment cycle comprises orally administering about 5 mg for between 9-19, preferably 14, consecutive days and said second treatment cycle comprises orally administering about 10 mg for at least 14 consecutive days; and
   (iii) during initiation of treatment, administering vericiguat in a dosing regimen comprising a first treatment cycle followed immediately by a second treatment cycle, wherein said first treatment cycle comprises orally administering 5 mg once daily with food for approximately two weeks and said second treatment cycle comprises orally administering 10 mg once daily with food.
19. A method of reducing the risk of cardiovascular death and heart failure hospitalization in a population of human patients with chronic heart failure, comprising administering to each human patient in the population vericiguat or a pharmaceutically acceptable salt thereof following a dosing regimen during initiation of treatment,
   wherein the dosing regimen comprises a first treatment cycle followed immediately by a second treatment cycle,
   wherein said first treatment cycle comprises orally administering about 5 mg, preferably 5 mg, for between 9-19, preferably 14, consecutive days and said second treatment cycle comprises orally administering about 10 mg, preferably 10 mg, for at least 14 consecutive days; and
   wherein the tolerability of the patient population is at least 90%,
20. The method of embodiment 19, wherein the tolerability of the patient population is at least 93%.
21. The method of embodiment 19 or 20, wherein tolerability is measured by the percentage of patients who complete the first treatment cycle with maximum one day interruption and without symptomatic hypotension of moderate to severe intensity within the first treatment cycle.
22. The method of embodiment 19, wherein tolerability is measured by the percentage of patients who complete the first treatment cycle without having an adverse event related to study intervention within the first treatment cycle.
23. The method of embodiment 22, wherein the adverse event is symptomatic hypotension of moderate to severe intensity.
24. The method of embodiment 19 or 20, wherein tolerability is measured by the percentage of patients who complete the first treatment cycle with continuous intervention intake or are able to restart intervention after temporary interruption of treatment.
25. The method of embodiment 24, wherein temporary interruption of treatment is defined as an interruption of treatment of 1 or 2 days.
26. The method of embodiment 19, 20, 21, 22, 23, or 24, wherein the human patients in the population have chronic heart failure with an ejection fraction of less than 45%.
27. A method of administering vericiguat or a pharmaceutically acceptable salt thereof, to a human subject in need thereof for use in treatment of cardiovascular and pulmonary disorders, comprising the following steps:
   administering a starting dose of 5 mg vericiguat once daily to a human subject in need thereof for use in treatment of cardiovascular and pulmonary disorders, doubling the dose after about 2 weeks to reach the target maintenance dose of 10 mg once daily, as tolerated by the patient,
   wherein for patients with a history of symptomatic hypotension the recommended starting dose is 2.5 mg vericiguat once daily, and the dose is doubled about every 2 weeks to reach the target maintenance dose of 10 mg once daily, as tolerated by the patient.
28. The method of embodiment 27, comprising the following steps:
   administering a starting dose of 5 mg vericiguat once daily to a human subject in need thereof for use in treatment of cardiovascular and pulmonary disorders, doubling the dose of vericiguat after about 2 weeks to reach the target maintenance dose of 10 mg once daily, as tolerated by the patient,
   wherein for patients with a recent history of symptomatic hypotension the recommended starting dose is 2.5 mg vericiguat once daily, and the dose is doubled about every 2 weeks to reach the target maintenance dose of 10 mg once daily, as tolerated by the patient,
   wherein recent history of symptomatic hypotension is defined as history of symptomatic hypotension 4 weeks before screening.
29. A method of administering vericiguat or a pharmaceutically acceptable salt thereof, to a human subject in need thereof for use in treatment of cardiovascular and pulmonary disorders, comprising the following steps:
   administering a starting dose of 5 mg vericiguat orally once daily with food to a human subject in need thereof for use in treatment of cardiovascular and pulmonary disorders, doubling the dose of vericiguat after about 2 weeks to reach the target maintenance dose of 10 mg once daily, as tolerated by the patient,
   wherein for patients with a history of symptomatic hypotension the recommended starting dose of vericiguat is 2.5 mg vericiguat orally once daily with food, and the dose is doubled about every 2 weeks to reach the target maintenance dose of 10 mg once daily, as tolerated by the patient.
30. The method of embodiment 29, comprising the following steps:
   administering a starting dose of 5 mg vericiguat orally once daily with food to a human subject in need thereof for use in treatment of cardiovascular and pulmonary disorders, doubling the dose of vericiguat after about 2 weeks to reach the target maintenance dose of 10 mg once daily, as tolerated by the patient,
   wherein for patients with a recent history of symptomatic hypotension the recommended starting dose of vericiguat is 2.5 mg vericiguat orally once daily with food, and the dose is doubled about every 2 weeks to reach the target maintenance dose of 10 mg once daily, as tolerated by the patient,
   wherein recent history of symptomatic hypotension is defined as history of symptomatic hypotension 4 weeks before screening.

### EXAMPLE

Chronic Heart Failure with Reduced Ejection Fraction

A Phase 2b open-label clinical study to evaluate the tolerability and safety of an initiation dose of 5 mg of Vericiguat in participants with chronic heart failure with reduced ejection fraction.

This was a 14-day study of initiation of treatment of vericiguat with a higher dose. The study design and procedures can be found under https://clinicaltrials.gov, study number NCT06195930.

Study Drug: Verquvo® vericiguat was approved for sale in the U.S. as 2.5, 5, and 10 mg tablets to reduce the risk of cardiovascular death and heart failure (HF) hospitalization following a hospitalization for heart failure or need for outpatient IV diuretics, in adults with symptomatic chronic HF and ejection fraction less than 45%. Vericiguat has the chemical name methyl {4,6-diamino-2-[5-fluoro-1-(2-fluorobenzyl)-1H-pyrazolo[3,4-b]pyridin-3-yl]pyrimidin-5-yl}carbamate and has the chemical structure shown above.

The overall study design is shown in FIG. 1. The patients had symptomatic chronic heart failure with reduced ejection fraction (HFrEF). This was a multi-center, single arm, open label study of vericiguat initiation at 5 mg in HFrEF patients with EF<45%. Participants underwent a screening visit to assess eligibility. Eligible participants waited a mandatory 2 weeks before returning for Visit 1 in order to be clinically and hemodynamically stable. At Visit 1 (V1), participants received vericiguat with directions to take a 5 mg tablet once daily for 2 weeks. Participants returned for Visit 2 (V2) after 2 weeks of treatment. There were unplanned healthcare visits for heart failure between Visits 1 and 2 at the discretion of the investigator.

Primary objective was to evaluate the tolerability of 5 mg as a starting dose of vericiguat. Endpoint: Treatment tolerability, defined as the completion of the two-week 5 mg dose without discontinuation of study intervention and without moderate to severe symptomatic hypotension between Visit 1 (V1) and Visit 2 (V2) Secondary objective 1 was to describe safety events of initiation of 5 mg dose. Endpoints: Any adverse event reported between V1 and V2. Absence of adverse events related to study intervention between V1 and V2.

Secondary objective 2 was to further evaluate the tolerability of 5 mg as a starting dose of vericiguat. Endpoint: Continuous intake of study intervention between V1 and V2 or restart of study intervention after any temporary interruption.

Inclusion and exclusion criteria aimed to ensure patients were hemodynamically stable and expected to tolerate treatment.

These were the patient inclusion criteria:
Patients with chronic HFrEF, LVEF<45% (assessed within 12 months before Visit 1 by any imaging method) and no subsequent LVEF measurement >45%.
Patients with (group 1) OR without (group 2) recent worsening HF event
   Group 1: History of chronic HF (NYHA class II symptomatic-IV) on GDMT with recent HF event within 6 months of screening or outpatient IV/SC diuretic use within 3 months before screening, OR
   Group 2: History of chronic HF (NYHA class II symptomatic-IV) on GDMT without recent HF event within 6 months of screening or outpatient IV/SC diuretic use within 3 months before screening.
SBP≥100 mmHg at screening and Visit 1 (pre-treatment)
No changes in GDMT dosing (including beta blockers, ACEI/ARBs, ARNI, MRAs, hydralazine-nitrate combinations, SGLT2 inhibitors, ivabradine, or oral diuretics)
   Within 4 weeks of screening for participants without a HF event <6 months prior to screening
   Within 2 weeks of screening for participants with a HF event <6 months prior to screening
   planned during study participation
No expected medical procedures to occur within 2 weeks before screening or during study participation.

These were the patient exclusion criteria:
History of symptomatic hypotension 4 weeks before screening, also defined as recent history of symptomatic hypotension.
Hemodynamic instability or hypovolemia within 4 weeks of screening and during the screening period
eGFR<15 mL/min/1.73 m² at within 30 days before start of treatment (Visit 1) or on chronic dialysis (for participants with multiple eGFR results during screening, the most recent value will be used to determine eligibility).
Cardiac conditions
Plan for heart transplant, or procedure requiring hospitalization
Concurrent or anticipated concomitant use of a PDE5 inhibitor during the study, or concurrent use of an sGC stimulator (e.g., riociguat)
Prior (within 2 weeks prior to screening) or anticipated concomitant administration of IV/SC diuretics or inotropes.

Dose changes in GDMT medications (RAS blockade, Beta-blocker, MRA, or SGLT2i) or oral diuretics for 2 weeks were discouraged, except when mandated by known side-effects for individual drug classes (e.g., hyperkalemia for MRA or angioedema for ACE-inhibitors/ARNI). Use of short and long-acting nitrates was permitted.

The study enrolled 125 patients, of which 19 did not complete screening. 106 patients with HFrEF (EF<45%, male 71.7%/female 28.3%, mean age 66.9, 50% each with and without recent worsening event, of which 47.2% on both ARNI and SGLT2i), were assigned to treatment (Tables 1 and 2).

TABLE 1
Disposition: flow of participants through
study phases (all participants)

| Number of participants | Total |
|---|---|
| Enrolled | 125 (100.0%) |
| Did not complete SCREENING | 19 (15.2%) |
| Primary reason | |
| OTHER | 1 (5.3%) |
| PHYSICIAN DECISION | 1 (5.3%) |
| SCREEN FAILURE | 14 (73.7%) |
| SUBJECT DECISION | 3 (15.8%) |
| Assigned to Treatment | 106 (100.0%) |
| Intervention never administered | 0 |
| Treated | 106 (100.0%) |
| TREATMENT phase | |
| Started | 106 (100.0%) |
| Completed | 102 (96.2%) |
| Did not complete | 4 (3.8%) |
| Primary reason | |
| ADVERSE EVENT | 4 (100.0%) |

Number of participants enrolled is the number of participants who signed informed consent. In case of rescreening, rescreened participants are counted once with their final enrollment.

TABLE 2
Demographics and other baseline characteristics of patients
treated in the study. 50% each with recent worsening event
(Group 1) and without recent worsening event (Group 2). “With
or without recent worsening HF event” being defined
as with or without HF event within 6 months of screening.

| | Vericiguat 5 mg |
|---|---|
| | N = 106 (100%) |
| Sex | |
| Female | 30 (28.3%) |
| Male | 76 (71.7%) |
| Race | |
| BLACK OR AFRICAN AMERICAN | 3 (2.8%) |
| NOT REPORTED | 1 (0.9%) |
| WHITE | 102 (96.2%) |
| Ethnicity | |
| HISPANIC OR LATINO | 39 (36.8%) |
| NOT HISPANIC OR LATINO | 67 (63.2%) |
| Age (YEARS) | |
| n | 106 |
| Mean (SD) | 66.9 (11.3) |
| Median | 67.5 |
| Min, Max | 32, 91 |
| Age Group 1 (EUDRACT) | |
| Adults (between 18 and 64 years) | 42 (39.6%) |
| From 65 to 84 years | 61 (57.5%) |
| 85 years and over | 3 (2.8%) |
| Age Group 2 | |
| <65 years | 42 (39.6%) |
| >=65 years | 64 (60.4%) |
| Screening Weight (kg) | |
| n | 106 |
| Mean (SD) | 84.758 (19.280) |
| Median | 82.000 |
| Min, Max | 50.00, 130.90 |
| Screening Height (cm) | |
| n | 106 |
| Mean (SD) | 169.190 (9.398) |
| Median | 170.000 |
| Min, Max | 147.00, 189.00 |
| a) History of HF event | |
| GROUP 1 | 53 (50.0%) |
| GROUP 2 | 53 (50.0%) |
| b) GDMT use | |
| SGLT2 INHIBITOR AND ARNI USE | 50 (47.2%) |

On Days 1-14, 5.0 mg Verquvo® vericiguat was administered orally once daily. The endpoint was tolerability based on need for treatment interruption between Days 1 and Day 14. Success of treatment was achieved since at least 90% patients continued through the 14-day titration without a medical need for interruption due to Verquvo® vericiguat.

Initiating treatment with vericiguat at 5 mg was well tolerated in the HFrEF population with and without recent worsening heart failure events treated with current GDMT.

Primary endpoint: 93.4% of participants completed the 2-week treatment period with the 5 mg dose of vericiguat without moderate to severe symptomatic hypotension and without temporary treatment interruptions exceeding 1 day.

Secondary endpoint 1: 90.6% of participants completed the 2-week treatment period without reported intervention-related adverse events (Table 3).

Secondary endpoint 2: 96.2% of participants completed treatment with continuous intervention intake or restarted study intervention after a temporary interruption (Table 3 and 4).

There were no major differences observed in the proportion of participants with and without recent worsening heart failure events who met the criteria for the primary and secondary endpoints (Table 3).

TABLE 3
Tolerability - Primary, secondary and sensitivity analyses.
Treatment tolerability of Vericiguat 5 mg (SAF).
Primary, secondary and sensitivity analyses:
Treatment tolerability of Vericiguat 5 mg (SAF)

| | OVERALL | GROUP 1 | GROUP 2 |
|---|---|---|---|
| Analysis type | N = 106 Num/Den(%) | N = 53 Num/Den(%) | N = 53 Num/Den(%) |
| Primary | 99/106 (93.4%) | 48/53 (90.6%) | 51/53 (96.2%) |
| Sensitivity | 102/106 (96.2%) | 50/53 (94.3%) | 52/53 (98.1%) |
| Secondary 1 | 96/106 (90.6%) | 49/53 (92.5%) | 47/53 (88.7%) |
| | 4 discontinuations | | |
| | for AE | | |
| Secondary 2 | 102/106 (96.2%) | 50/53 (94.3%) | 52/53 (98.1%) |

Number of participants in denominator is number of participants in SAF in the respective subgroup
Primary: Participants who completed the two-week treatment with maximum one day interruption and without symptomatic hypotension of moderate to severe intensity between Visit 1 and Visit 2.
Sensitivity: Participants who completed the two-week treatment with maximum two days interruption and without symptomatic hypotension of moderate to severe intensity between Visit 1 and Visit 2
Secondary 1: Participants who did not have an AE related to study intervention between Visit 1 and Visit 2.
Secondary 2: Participants who completed the two-week treatment with continuous intervention intake or were able to restart intervention after temporary interruption.
Group 1: Participants with recent worsening HF event, Group 2: Participants without recent worsening HF event.

TABLE 4
Duration of treatment interruptions/delay (SAF - Safety Analysis
Set, all participants assigned to investigational intervention who
were exposed to study intervention at least once.)
Treatment interruptions (SAF)

| Duration of | Total Duration of | Vericiguat 5 mg |
|---|---|---|
| Interruption/Delay | Interruption/Delay (days) | N = 106 (100%) |
| | n | 106 |
| | Mean (SD) | 0.1 (0.3) |
| | Median | 0.0 |
| | Min, Max | 0, 2 |
| | 0 | 102 (96.2%) |
| | 1 | 1 (0.9%) |
| | 2 | 3 (2.8%) |

TABLE 5
Safety. Adverse events: overall summary of number of participants (SAF).
Adverse events: overall summary of number of participants (SAF)

| | Vericiguat 5 mg |
|---|---|
| | N = 106 (100%) |
| Number (%) of participants with adverse events | |
| Any AE | 14 (13.2%) |
| Maximum intensity for any AE | |
| MILD | 9 (8.5%) |
| MODERATE | 4 (3.8%) |
| SEVERE | 1 (0.9%) |
| Any intervention-related AE | 10 (9.4%) |
| Maximum intensity for intervention-related AEs | |
| MILD | 8 (7.5%) |
| MODERATE | 2 (1.9%) |
| Any AE related to procedures required by the protocol | 0 |
| Any AE leading to discontinuation of intervention | 4 (3.8%) |
| Any AE leading to dose modification of study intervention[a] | 6 (5.7%) |
| Any AE of special interest | 6 (5.7%) |
| Any SAE | 1 (0.9%) |
| SAE with fatal outcome | 0 |
| Life-threatening SAE | 0 |
| SAE requiring hospitalization | 1 (0.9%) |
| SAE resulting in substantial disruption of normal life functions | 0 |
| Congenital anomaly or birth defect | 0 |
| Other | 0 |
| Any intervention-related SAE | 0 |
| Any SAE related to procedures required by the protocol | 0 |
| Any SAE leading to discontinuation of intervention | 0 |

[a]Modifications include delays, interruptions and reductions. An SAE can fulfill more than one reason for seriousness.

The results of the study allow an update of the respective Verquvo® vericiguat country drug labels with a recommended starting dose of Verquvo® vericiguat of 5.0 mg once daily to reach the target maintenance dose of 10 mg, as tolerated by the patient. This will provide the physicians the choice of safely initiating vericiguat at a higher dose, thus reducing the titration steps and facilitating the attainment of the 10 mg target dose. The goal is to reduce complexity and burden to the patients.

The recommended starting dose is 5 mg vericiguat once daily. The dose should be doubled after approximately 2 weeks to reach the target maintenance dose of 10 mg once daily, as tolerated by the patient. For patients with a history of symptomatic hypotension the recommended starting dose is 2.5 mg vericiguat once daily. Double the dose approximately every 2 weeks to reach the target maintenance dose of 10 mg once daily, as tolerated by the patient. Method of administration: For oral use. Verquvo® should be taken with food.

The recommended starting dose is 5 mg vericiguat once daily. The dose should be doubled after approximately 2 weeks to reach the target maintenance dose of 10 mg once daily, as tolerated by the patient. For patients with a recent history of symptomatic hypotension the recommended starting dose is 2.5 vericiguat once daily. Double the dose approximately every 2 weeks to reach the target maintenance dose of 10 mg once daily, as tolerated by the patient. Method of administration: For oral use. Verquvo® should be taken with food.

The recommended starting dose of Verquvo® is 5 mg orally once daily with food. Double the dose of Verquvo® after approximately 2 weeks to reach the target maintenance dose of 10 mg once daily, as tolerated by the patient. For patients with a history of symptomatic hypotension the recommended starting dose of Verquvo® is 2.5 mg vericiguat orally once daily with food. Double the dose approximately every 2 weeks to reach the target maintenance dose of 10 mg once daily, as tolerated by the patient.

The recommended starting dose of Verquvo® is 5 mg orally once daily with food. Double the dose of Verquvo® after approximately 2 weeks to reach the target maintenance dose of 10 mg once daily, as tolerated by the patient. For patients with a recent history of symptomatic hypotension, the recommended starting dose of Verquvo® is 2.5 mg orally once daily with food. Double the dose approximately every 2 weeks to reach the target maintenance dose of 10 mg once daily, as tolerated by the patient.

Recent history of symptomatic hypotension within the meaning of the disclosure is defined as history of symptomatic hypotension 4 weeks before screening.

Numerous modifications to the present disclosure will be apparent to those skilled in the art in view of the foregoing description. Accordingly, this description is to be construed as illustrative only and is presented for the purpose of enabling those skilled in the art to make and use the disclosure. The exclusive rights to all modifications which come within the scope of the appended claims are reserved. All patents and documents referenced herein are hereby incorporated herein in their entireties by reference.

### DESCRIPTION OF THE FIGURES

FIG. 1: study design. S=screening, A=assignment to treatment; EoS=end of study; D=day; V=visit.

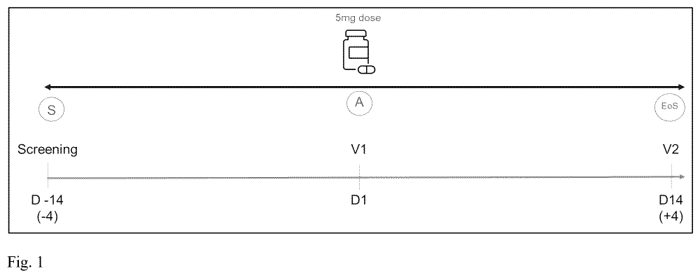

We claim:

1. A method of administering vericiguat or a pharmaceutically acceptable salt thereof, to a human subject in need thereof for use in treatment of cardiovascular and pulmonary disorders, comprising the following steps: during initiation of treatment, administering vericiguat, or a pharmaceutically acceptable salt thereof, in a dosing regimen comprising a first treatment cycle followed immediately by a second treatment cycle, wherein said first treatment cycle comprises orally administering about 5 mg for between 9-19 consecutive days and said second treatment cycle comprises orally administering about 10 mg for at least 14 consecutive days.

2. The method of claim 1, wherein said first treatment cycle comprises orally administering at least 5 mg for between 9-19 consecutive days and said second treatment cycle comprises orally administering at least 10 mg for at least 14 consecutive days.

3. (canceled)

4. The method of claim 1, wherein said first treatment cycle comprises orally administering at least 5 mg for 14 consecutive days and said second treatment cycle comprises orally administering at least 10 mg for at least 14 consecutive days.

5. (canceled)

6. (canceled)

7. (canceled)

8. (canceled)

9. (canceled)

10. The method of claim 1, wherein the human subject has symptomatic chronic heart failure and ejection fraction less than 45% upon initiation of vericiguat administration.

11. The method of claim 1, wherein the treatment of cardiovascular and pulmonary disorders results in reduced risk of cardiovascular death or heart failure hospitalization.

12. The method of claim 1, wherein the treatment of cardiovascular and pulmonary disorders results in a reduced risk of a composite endpoint of cardiovascular death or heart failure hospitalization.

13. The method of claim 1, wherein vericiguat is administered and upon initiation of vericiguat administration the human subject has symptomatic chronic heart failure and ejection fraction less than 45% and has had no hospitalization within six months for heart failure and no outpatient IV or SC diuretic use within three months.

14. The method of claim 1, wherein vericiguat is administered and upon initiation of vericiguat administration the human subject has symptomatic chronic heart failure and ejection fraction less than 45% and has had hospitalization within six months for heart failure or outpatient IV or SC diuretic use within three months.

15. The method of claim 1, further comprising administering a further therapeutic agent selected from the group consisting of beta-blockers, angiotensin-converting enzyme inhibitors (ACEI), angiotensin receptor blockers (ARB), angiotensin receptor-neprilysin inhibitors (ARNI), mineralocorticoid receptor antagonists (MRA), and sodium-glucose cotransporter-2 inhibitors (SGLT2i).

16. The method of claim 1, further comprising administering the further therapeutic agents angiotensin receptor-neprilysin inhibitors (ARNI) and sodium-glucose cotransporter-2 inhibitors (SGLT2i).

17. The method of claim 1, wherein vericiguat is administered.

18. (canceled)

19. A method of reducing the risk of cardiovascular death and heart failure hospitalization in a population of human patients with chronic heart failure, comprising administering to each human patient in the population vericiguat or a pharmaceutically acceptable salt thereof following a dosing regimen during initiation of treatment,
wherein the dosing regimen comprises a first treatment cycle followed immediately by a second treatment cycle,
wherein said first treatment cycle comprises orally administering about 5 mg, preferably 5 mg, for between 9-19, preferably 14, consecutive days and said second treatment cycle comprises orally administering about 10 mg, preferably 10 mg, for at least 14 consecutive days; and
wherein the tolerability of the patient population is at least 90%.

20. (canceled)

21. (canceled)

22. (canceled)

23. (canceled)

24. (canceled)

25. (canceled)

26. The method of claim 19, wherein the human patients in the population have chronic heart failure with an ejection fraction of less than 45%.

27. A method of administering vericiguat or a pharmaceutically acceptable salt thereof, to a human subject in need thereof for use in treatment of cardiovascular and pulmonary disorders, comprising the following steps:
administering a starting dose of 5 mg vericiguat once daily to a human subject in need thereof for use in treatment of cardiovascular and pulmonary disorders, doubling the dose after about 2 weeks to reach the target maintenance dose of 10 mg once daily, as tolerated by the patient,
wherein for patients with a history of symptomatic hypotension the recommended starting dose is 2.5 mg vericiguat once daily, and the dose is doubled about every 2 weeks to reach the target maintenance dose of 10 mg once daily, as tolerated by the patient.

28. (canceled)

29. A method of administering vericiguat or a pharmaceutically acceptable salt thereof, to a human subject in need thereof for use in treatment of cardiovascular and pulmonary disorders, comprising the following steps:
administering a starting dose of 5 mg vericiguat orally once daily with food to a human subject in need thereof for use in treatment of cardiovascular and pulmonary disorders, doubling the dose of vericiguat after about 2 weeks to reach the target maintenance dose of 10 mg once daily, as tolerated by the patient,
wherein for patients with a history of symptomatic hypotension the recommended starting dose of vericiguat is 2.5 mg vericiguat orally once daily with food, and the dose is doubled about every 2 weeks to reach the target maintenance dose of 10 mg once daily, as tolerated by the patient.

30. (canceled)
